(19) United States
(12) Patent Application Publication
(10) Pub. No.: US 2023/0248287 A1
(43) Pub. Date: Aug. 10, 2023

(54) HYPERACTIVITY-IMPULSIVITY-IRRITATBILITY-DISINHIBITION-AGGRESSION-AGITATION (HIIDAA) REDUCTION AND MANAGEMENT DEVICE, AND METHOD OF USE

(71) Applicant: Soul Care Innovations, Inc., North Kingstown, RI (US)

(72) Inventors: Marybeth Marshall, Providence, RI (US); Yasmin Karasu Benyes, Providence, RI (US)

(73) Assignee: Soul Care Innovations, Inc., North Kingstown, RI (US)

(21) Appl. No.: 18/159,475

(22) Filed: Jan. 25, 2023

Related U.S. Application Data
(60) Provisional application No. 63/308,092, filed on Feb. 9, 2022.

(51) Int. Cl. A61B 5/16; A61B 5/00; G16H 40/67; G16H 50/70

(52) U.S. Cl. CPC A61B 5/165; A61B 5/0022; A61B 5/742; G16H 40/67; G16H 50/70

(57) ABSTRACT

A motion platform combines oscillating single-axis or bi-axial motion and patient biological and behavioral feedback mechanisms for reducing HIIDAA in people experiencing neurological imparities. The platform is driven by actuators that provide single-axis or bi-axial motion. The platform has a planar upper surface on which a wheelchair, chair or other resting furniture can be positioned. The platform motion is actuated based on facial expression and bodily movement recognition feedback, heart rate feedback, standing and fall detection, and/or manual remote control from a wireless or internet enabled device. Noise feedback may also be provided as an input. The device actuation may include oscillating motion to simulate rocking. Music may also be output to further help manage the reduction of HIIDAA of the individual. Reinforcement and deep learning algorithms also use optimal time-dependent actuation profiles based on real-time inputs and a database of behavior characteristics of the patient.

### CROSS-REFERENCE TO RELATED APPLICATIONS

This Non-Provisional patent application claims the benefit of U.S. Provisional Patent Application No. 63/308,092 filed Feb. 9, 2022 and entitled Hyperactivity-Impulsivity-Irritability-Disinhibition-Aggression-Agitation (HIIDAA) Reduction And Management Device And Method Of Use, the entire contents of which is incorporated herein by reference.

### FIELD OF THE INVENTION

This invention relates generally to systems and methods for treating, managing and/or improving the psychological and emotional well-being of people experiencing Alzheimer's Disease (AD), Autism Spectrum Disorders (ASD), and Behavioral Psychological Symptoms of Dementia (BPSD) and more specifically to systems and methods for treating, managing and/or improving Hyperactivity-Impulsivity-Irritatbility-Disinhibition-Aggression-Agitation (HIIDAA) which encompasses all states of AD, ASD and BPSD.

### BACKGROUND OF THE INVENTION

Dementia is characterized by a progressive decline in cognitive functions and the ability to execute activities of daily living. Severe behavioral and psychological symptoms of dementia (BPSD) are nearly universal in patients. For example, in Alzheimer's disease (AD), which represents 60-80% of all dementias, over 90% of patients display BPSD, including depression, anxiety, apathy, agitation and aggression, disinhibition, delusions, hallucinations, irritability, and emotional lability, euphoria, and aberrant motor, sleep, and eating behaviors. Overall, BPSD is associated with decreased quality of life, increased cognitive and functional decline, greater likelihood of institutionalization, and heightened risk of mortality. Moreover, these symptoms are correlated with greater direct and indirect costs for patient care as well as significantly higher caregiver burdens.

Similarly, Autism Spectrum Disorder (ASD) is a neurodevelopmental disorder characterized by markedly impaired social interaction, impaired communication, and restricted/repetitive patterns of behavior, interests, and activities. In addition to challenges caused by core symptoms of the disorder, maladaptive behaviors such as aggression can be associated with ASD and can further disrupt functioning and quality of life. As most individuals with ASD will spend the majority of their lives with ASD as adults, there is a compelling need for effective treatments for these maladaptive behaviors in adults in order to minimize adverse outcomes, which, in the case of aggression, can be dangerous for caregivers.

One of the domains which can describe all states of BPSD and ASD is Hyperactivity-Impulsivity-Irritability-Disinhibition-Aggression-Agitation (HIIDAA). The HIIDAA domain represents one of the most difficult sets of symptoms to manage in AD or ASD and accounts for much of the burden for caregivers and hospital staff.

### SUMMARY OF THE INVENTION

There are no known HIIDAA management devices or systems which are based on repetitive motion and sound/music therapy systems combined with real-time behavioral observation, data collection and real-time therapy adaptations to reduce HIIDAA behaviors.

The present disclosure focuses on calming the HIIDAA domain from non-pharmacological interventions.

Psychological and emotional well-being improvements have been associated with repetitive motion therapies, such as patients being rocked in a rocking chair. This oscillating movement is used for reducing agitation in patients with neurological impairments, such as those with dementia or Alzheimer's Disease. It is an object of this disclosure to make these non-pharmaceutical methods for calming patients more easily accessible to all individuals by automating this rocking motion based on individual needs.

A motion platform combines oscillating single-axis or bi-axial motion and patient biological and behavioral feedback mechanisms for reducing HIIDAA in people experiencing neurological imparities. The platform is driven by actuators that provide single-axis or bi-axial motion. The platform has a planar upper surface on which a wheelchair, chair or other resting furniture can be positioned. The platform can be locked in place for loading and unloading a patient onto the platform.

Automated feedback and rocking can provide automatic calming of agitation symptoms through the use of video based feedback mechanisms which monitor facial expression recognition and repetitive physical movements and other biological feedback mechanisms such as monitoring increased heart rate. In some environments, noise or repetitive speech may be monitored and used as feedback inputs.

Responsive to the inputs, the system may be actuated to create repetitive oscillating motions that can stimulate the vestibular system and also increase relaxation in people while decreasing aggressive behaviors. Literature shows that rocking can be used as a relaxation mechanism to increase positive emotions and decrease heart rates in patients experiencing neurological imparities, such as dementia. While rocking has been explored previously, the automated rocking platform and integrated feedback mechanisms are a unique combination that will help to customize this experience for each individual while also making the technology accessible to everyone despite any physical or physiological limitations they may have since this platform is suitable for any type of chair.

Configurations herein are based on a gliding swing motion where the platform moves on a planar biaxial stage in an oscillation motion. The platform is designed in a way so that any chair a person may sit in, including those best suited for patients with neurological impairments, can fit onto the platform and be locked into place to ensure the persons' safety. The platform can then be actuated manually or automatically via a feedback mechanism. Once the platform is engaged, it will move until triggered to stop manually or via a feedback mechanism. Conventional approaches to this rocking motion require the person to rock themselves in the chair or the use of an aid to move the chair in the oscillating motion. This method instead automates that process making it accessible to all patients and allowing aid to focus on other tasks.

The disclosed approach is particularly beneficial in that it also incorporates feedback mechanisms for the patient to signal when the platform should be activated or not to reduce agitation. This includes facial and body recognition, heart rate monitoring, increased movement, and noise volume monitoring.

Reinforcement learning algorithms are used to learn which feedback mechanisms best indicate an agitated behavior state and therefore are needed to trigger the use of the platform to help relax the patient.

### BRIEF DESCRIPTION OF THE DRAWINGS

These and other advantages of the present invention will become more readily apparent upon reading the following detailed description and upon reference to the drawings in which

FIG. 1 is a perspective view of an exemplary embodiment of a HIIDAA reduction and management system including an depiction of an exemplary dementia patient sitting in a wheelchair placed on the device platform;

FIG. 2A is a perspective view of the system platform;

FIG. 2B is an exploded view thereof;

FIG. 2C is a perspective view of the internal X and Y axis working components of the platform;

FIG. 2D is a perspective view of the Y-axis motion actuator mounted on a bridge support;

FIG. 3A is block diagram of the electronic communication elements, input components and output components of the system;

FIG. 3B is a block diagram of an exemplary system microcontroller (Rasberry Pi);

FIG. 3C is an illustration of an exemplary patient worn biometric health sensor band;

FIG. 3D is a block diagram of the health sensor band;

FIG. 4 is a block diagram of the overall software control system including inputs and outputs;

FIGS. 5A-5C are flow diagrams of the facial expression and agitation detection module;

FIGS. 6A-6B are flow diagrams of a head movement detection module;

FIG. 7 is a flow diagram of a head movement detection and control module;

FIG. 8A is a flow diagram of an elevated heart rate detection module;

FIG. 8B is a flow diagram of an excessive rapid movement detection module;

FIG. 9 is a flow diagram of a multi-input behavioral state control module;

FIG. 10 is a flow diagram of a stand/fall detection module;

FIG. 11A shows various operating positions of the platform, including a locked loading position, a home position and a center position; and

FIG. 11B are flow diagrams of the start, motion and stop sequences when employing the solenoid locking safety system.

### DETAILED DESCRIPTION OF EXEMPLARY EMBODIMENTS

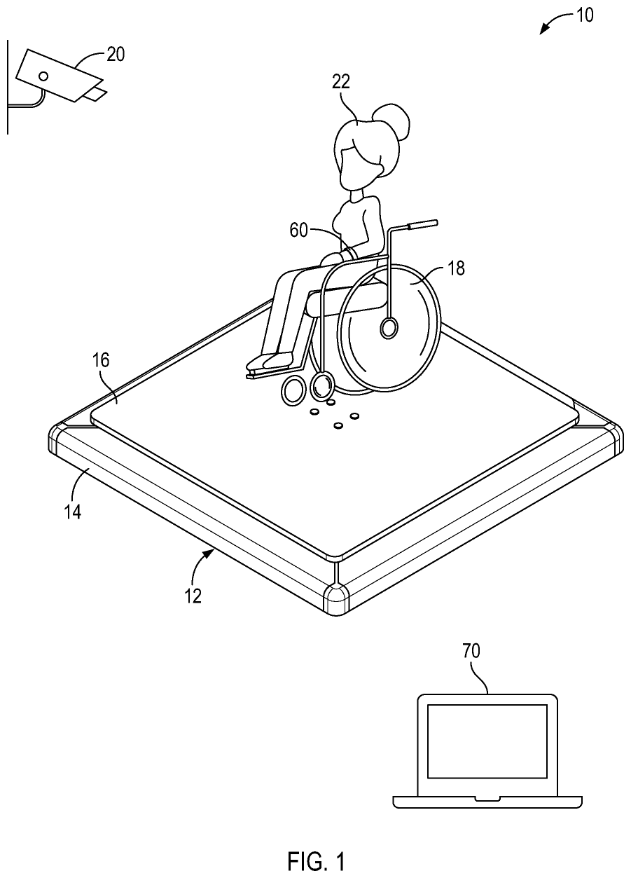

Referring now to the drawings, an exemplary embodiment of the present therapeutic system is generally indicated at 10 in FIGS. 1-10.

The therapeutic system for managing behavioral symptoms of neurological disorders generally comprises a motion device or apparatus 12 comprising a base 14 and platform 16 movable relative to the base 14, the platform 16 having an upper surface configured to receive and support a seat 18 thereon, a camera device 20 configured to acquire a continuous sequence of images of a patient 22 seated on the platform 16, and further comprises a feedback and control system generally indicated at 24.

The seat 18 may comprise any type of chair or seat or other patient support which is configured to comfortably support the patient on the platform. The exemplary illustration shows a wheelchair, but the term “seat” should not be considered limiting. The seat should be capable of being locked in a stationary position upon the platform, i.e. where a wheelchair or other movable chair or support includes locking wheels. Otherwise, the platform 16 and/or seat 18 may be provided with other interlocking or securing apparatus on the upper surface to secure the seat 18 in position on the platform 16.

The camera 20 may comprise any video device which is capable of providing a continuous video stream at a given frame rate for sequential or periodic analysis of images for changes in patient behavior. The video stream may be transmitted through a wired or wireless connection as appropriate for the environment. Such video camera devices are well known in the art and will not be described further.

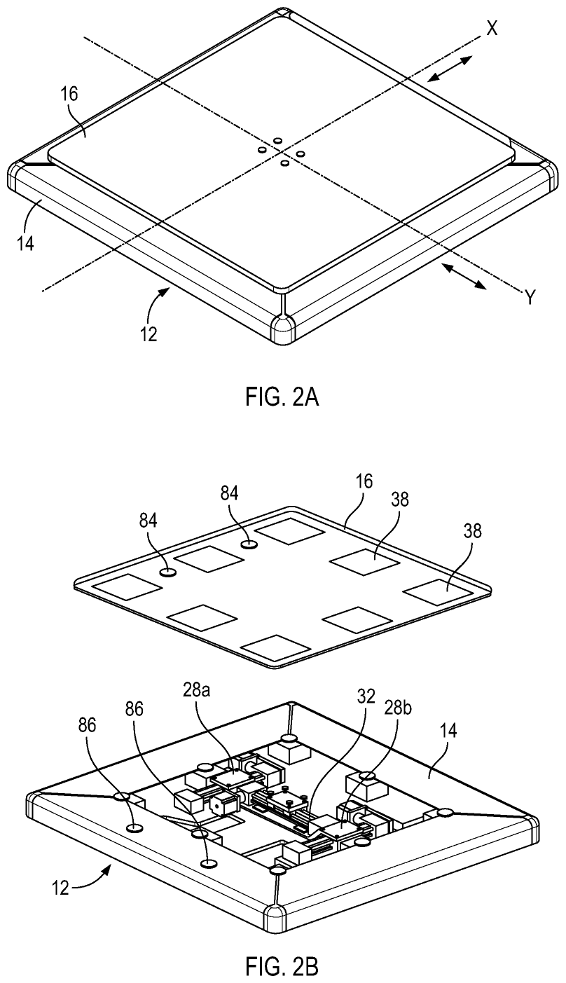

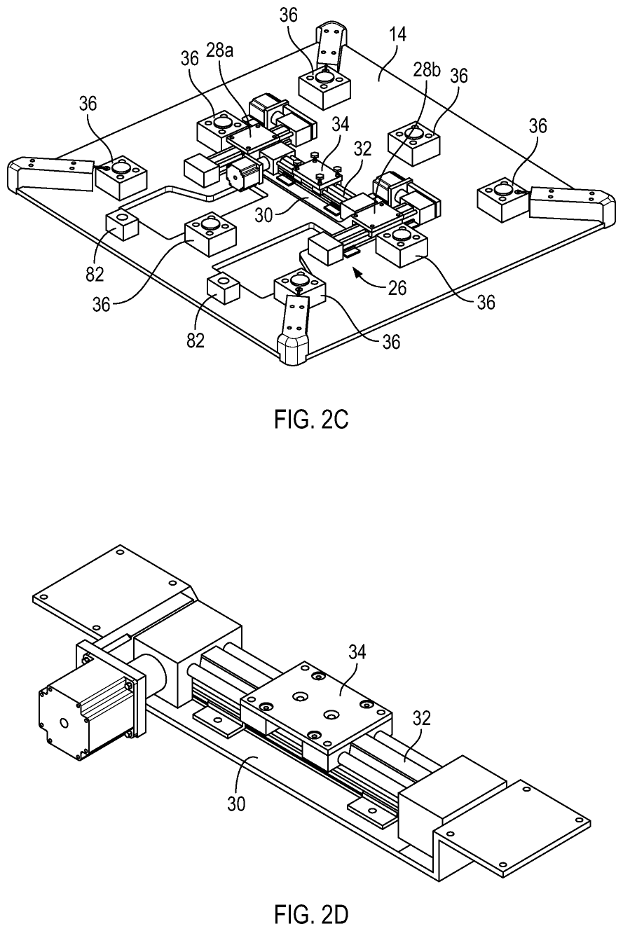

The motion device 12 further comprising a motion actuation apparatus 26 connected between the base 14 and the platform 16 which effective for either single-axis or biaxial oscillating motion of the platform 16 relative to the base 14. The exemplary embodiment may provide therapeutic oscillating movement of the platform along X and Y planes as illustrated in FIG. 2A. Oscillating movement can be provided at a range of frequencies which may be between 0.1 Hz and 1.0, but preferably should at least be greater than 0.3 Hz.

Turning to FIGS. 2A-2D, the motion actuation apparatus 26 is illustrated in more detail.

The motion actuation apparatus 26 may comprise two (2) spaced parallel linear actuator stages 28A,28B operating in the X plane of movement. A support bridge 30 extends between the mount blocks of the two X-axis stages 28A,2B which supports a another linear actuator stage 32 operating in the Y-axis. A platform anchor block 34 is mounted on top of the Y-axis stage 32. The platform 16 is in turn mounted to the platform anchor block 34. Each linear actuator stage 28A,28, 30 includes its own drive motor which is operated by the feedback and control system 24.

Further supporting the peripheral edge portions of the platform 16 relative to the base 14 are a plurality of ball bearing supports 36A-36H. These bearings 36A-36H sit beneath a like plurality of low friction slide plates 38A-38H mounted to the underside of the platform 16. This support system better distributes weight across the base, provides quiet motion and limits strain on the actuator motors.

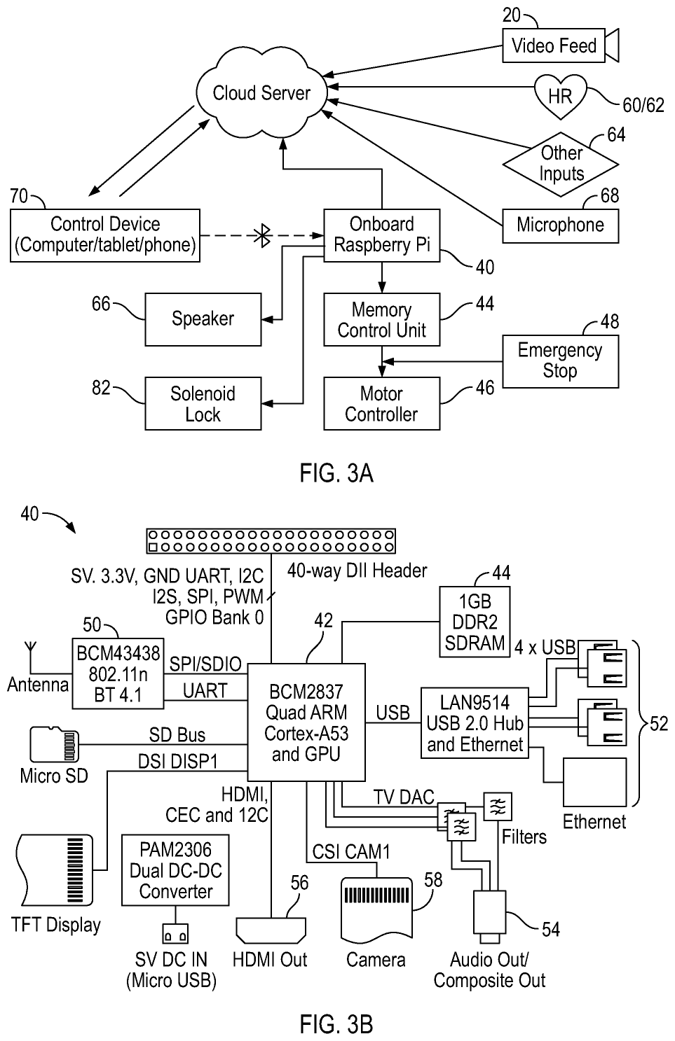

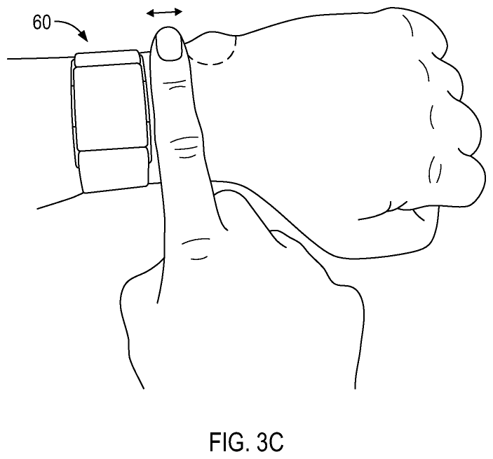

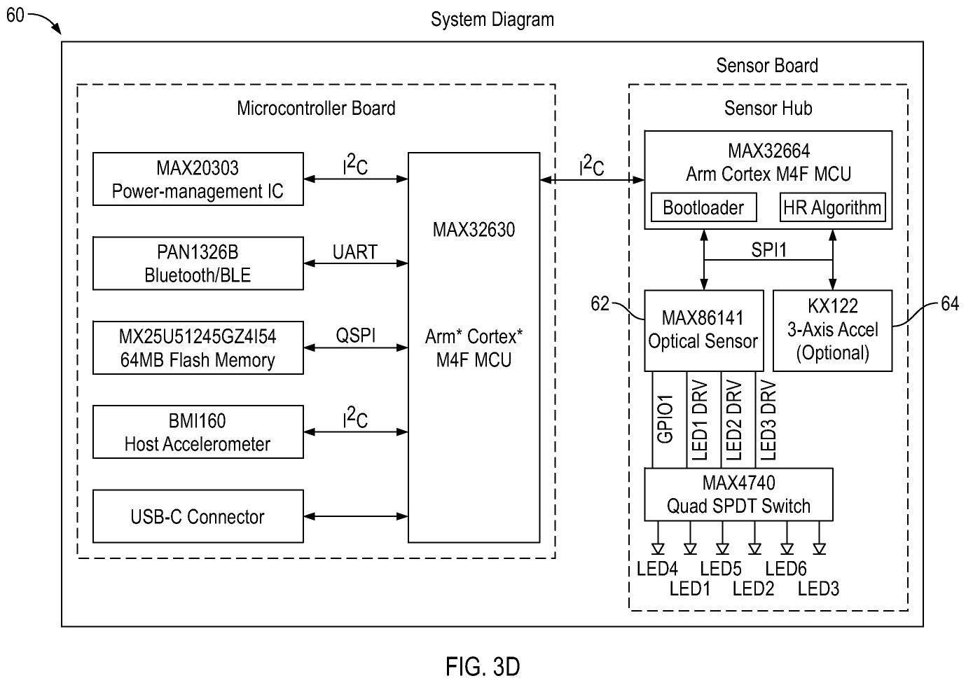

Turning to FIG. 3A, there is illustrated a block diagram of the elements of the control and feedback system 24. The system 24 generally includes camera 20, a microcontroller 40 including a CPU 42 programmed with an analytics engine and a memory 44 housing a data store, and a motor controller 46 which drives the X and Y axis actuator stages 28, 30. An emergency stop switch 48 may be provided on the platform.

Referring to FIG. 3B, the microcontroller 40 may comprise a Raspberry Pi microcontroller system including CPU 42, memory 44, wireless communication port(s) (Bluetooth/WiFi) 50, wired communication port(s) (USB/Ethernet) 52, audio output port 54, video output port (HDMI) 56, and a video/camera input port 58. Input from camera 20 may be provided wirelessly (Bluetooth or Wifi) or wired.

The feedback and control system operates through video feedback of the patient in conjunction with an analytics engine which analyzes the video feed for changes in emotional state of the patient being monitored. The data store (memory) 44 is encoded with content including a plurality of predetermined motion control profiles associated with managing predetermined agitated emotional states of a patient. The analytics engine programmed within microcontroller 40 is connected with the camera 20, the data store 44 and the motor controller 46 and is programmed to receive a continuous sequence of images from the camera, analyze facial expression, head movements and/or physical movements within the sequential images to determine in real-time an emotional state of the patient, and automatically alter operation of the motion actuation apparatus based on the emotional state of the patient.

In some embodiments the data store is further encoded with content including previously captured images of the patient in normal emotional states and agitated emotional states for real-time comparison.

To provide additional feedback inputs to the microcontroller, a wrist worn wireless health monitoring device 60 may be provided, such as Maxim Integrated—MAXREDDES103 Health Sensor Band (See FIG. 3C). A block diagram of the wrist worn band 60 is illustrated in FIG. 3D. Health sensor bands of the type contemplated are well known in the art and the details thereof will not be described further other than the needed outputs for the current system implementation.

The wrist band 60 may include an optical sensor 62 which enables patient heart rate detection and an accelerometer 64 for rapid movement or fall detection. The wrist band 60 may be wirelessly connected with the microcontroller 46 of the feedback and motion control system for input to the analytics engine. In operation, the data store is further encoded with content including acceptable and out of range heart rate profiles wherein the output of the heart rate detection device 60 is used as a further input to the analytics engine for analyzing and determining in real-time an emotional state of the patient.

Similarly, the accelerometer device 64 may be wirelessly connected with the feedback and motion control system, wherein the data store is still further encoded with content including acceptable and out of range motion profiles, and/or fall detection profiles. Rapid agitated movement of the patients arms may be further indicative of an agitated state. An output of the accelerometer device 64 may thus be used as a further input to the analytics engine for analyzing and determining in real-time an emotional state of the patient.

As a further means for therapeutic effect, the system 10 may further comprise an audio output device, such as a speaker 66 connected with the feedback and control system. In this regard, the data store may be further encoded with auditory content, such as music. In certain predetermined therapy profiles, the analytics engine may automatically alter or trigger operation of speaker device to output the auditory content based on the emotional state of the patient.

In some embodiments, the therapeutic system 24 may comprise a microphone 68 connected with the feedback and motion control system wherein an output of the microphone is used as a further input to the analytics engine for analyzing and determining in real-time an emotional state of the patient. In some embodiments, such as nursing homes, the ambient background noise may be too excessive to isolate patient vocal output to be useful as an input. However, in more private settings, the microphone could be used to detect agitated vocalizations, chanting etc. which can also be indicative of agitated emotional states. Additionally, the data store may be encoded with further content including previously captured recordings of the patient in normal emotional states and agitated emotional states for comparison and use in analyzing the patient's real-time emotional state.

The system further comprises a control device 70, such as a computer, tablet, cell phone, etc., which communicates (wired or wireless) with the microcontroller 40 through a graphical user interface installed on the control device for operation of the system 10. The control device 70 may include automated programming sequences, and may include manual inputs for time duration, oscillation frequency of one or both axes of movement, enabling of selected inputs, etc. and an emergency stop override.

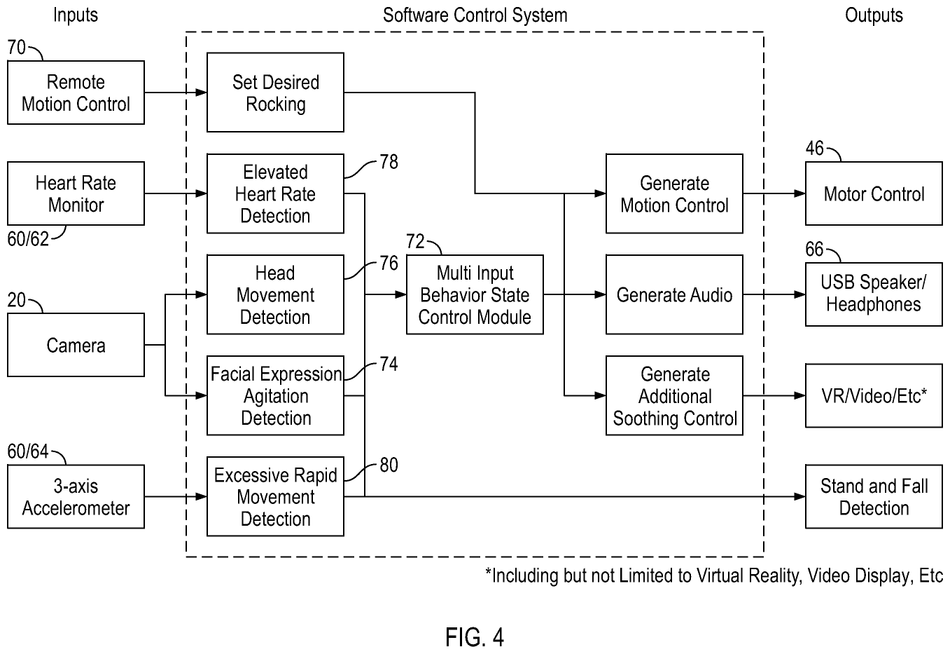

The overall software control scheme and various algorithms involved with analyzing the incoming images can be found in FIGS. 4-10.

FIG. 4 illustrates that the software system is composed of three parts: inputs, control systems, and outputs. Video camera 20, heart rate sensor 60/62, and accelerometer 60/64 are inputs directly from the patient. Manual control can be implemented by the caregiver to adjust the oscillation speed or other parameters as needed. The Software control system uses the multiple inputs to determine HIIDAA based on a plurality of corresponding state detection algorithms. A multi-input behavioral state control module 72 uses a weighted preset or learned voting method based on the results of the various detection algorithms (facial expression 74, head movement 76, elevated heart rate 78 and excessive movement 80) to generate control signals which trigger motion, audio, and/or additional soothing features that may be included, for example, virtual reality, in the output devices, i.e. motor control 46, speaker 66, etc. Accelerometer inputs can also be used as a safety measure to detect if the patient has fallen to stop motion and notify the caregiver.

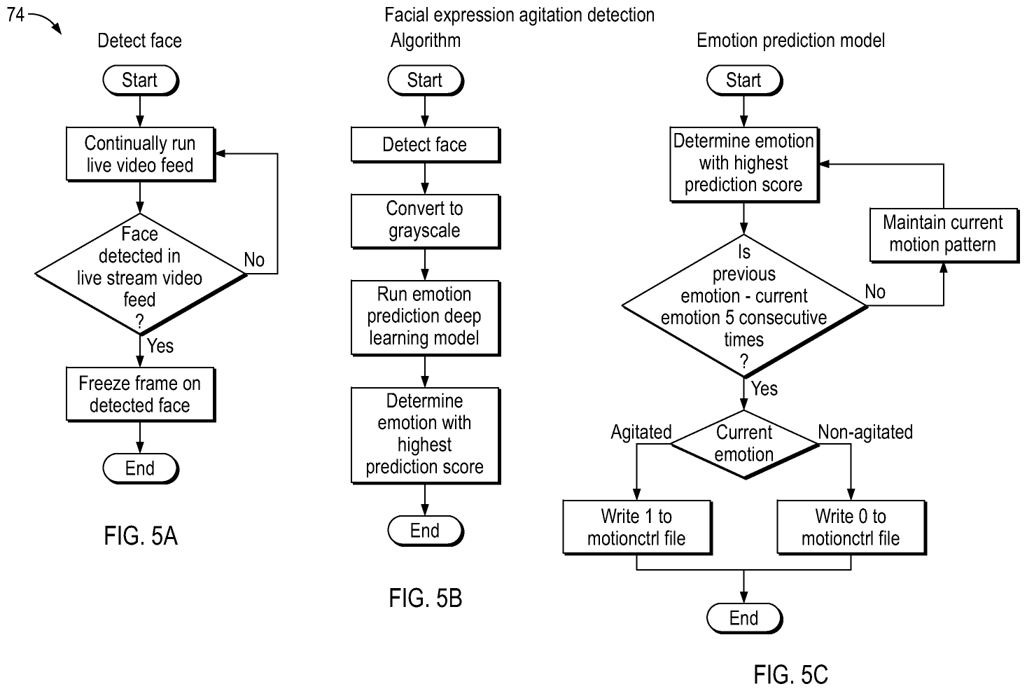

FIGS. 5A-5C are a detailed explanation of a facial expression detection algorithm 74. FIG. 5A describes the algorithm 74 used to detect a face based on the continuous live video feed from the camera 20. FIG. 5B processes the freeze-frame of the detected face to determine the patient's agitation state. Finally, FIG. 5C uses the current and past predictions to determine a continuous state of HIIDAA and returns its vote to the multi-input state control module.

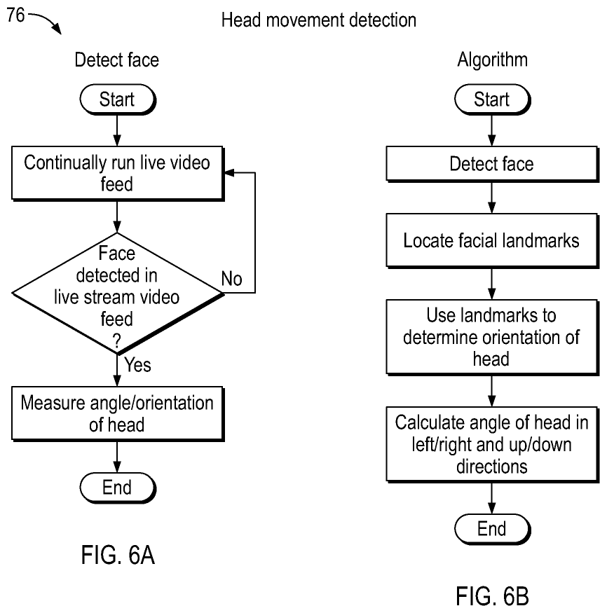

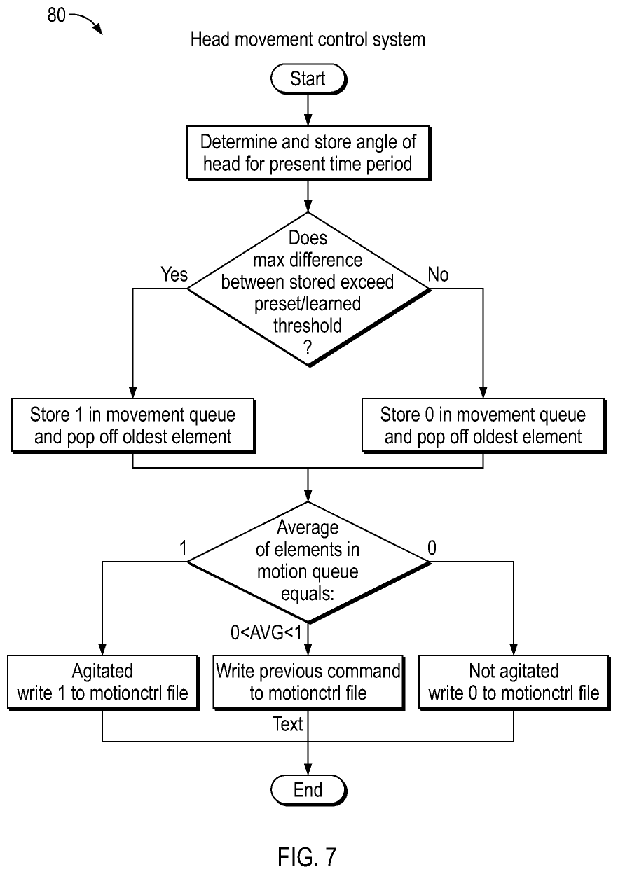

FIGS. 6A-6B are a detailed explanation of head movement algorithm 76 to detect repeated excessive head movements in a set period of time by tracking and storing consecutive flagged movements that have been determined to be above a preset or learned threshold. FIG. 6A provides a real-time measurement of the orientation and angle of the patient's head by using facial landmark detection as seen in FIG. 6B. FIG. 7 uses the current and past predictions to determine a continuous state of HIIDAA and returns it's a vote to the multi-input state control 72.

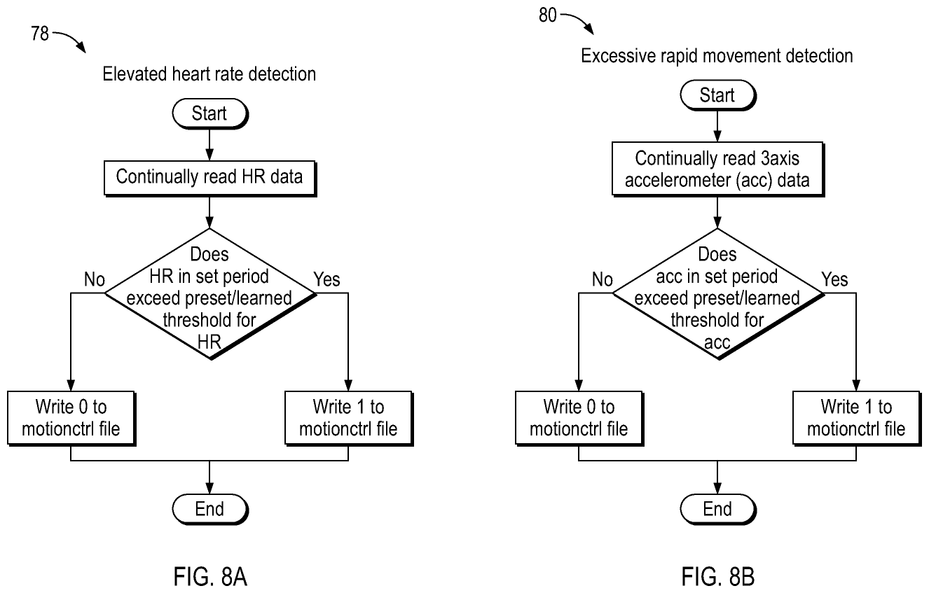

FIGS. 8A-8B corresponds to the heart rate detection algorithm model 78 which designed to identify increases which exceed a preset or learned threshold. Similarly, the heart rate tracking algorithm tracks heart rate over a period of time and flags elevated heart rates that exceed a preset or learned threshold. Each algorithm seeks to determine if the patient is in an agitated states and returns a respective votes to the multi-input state control 72.

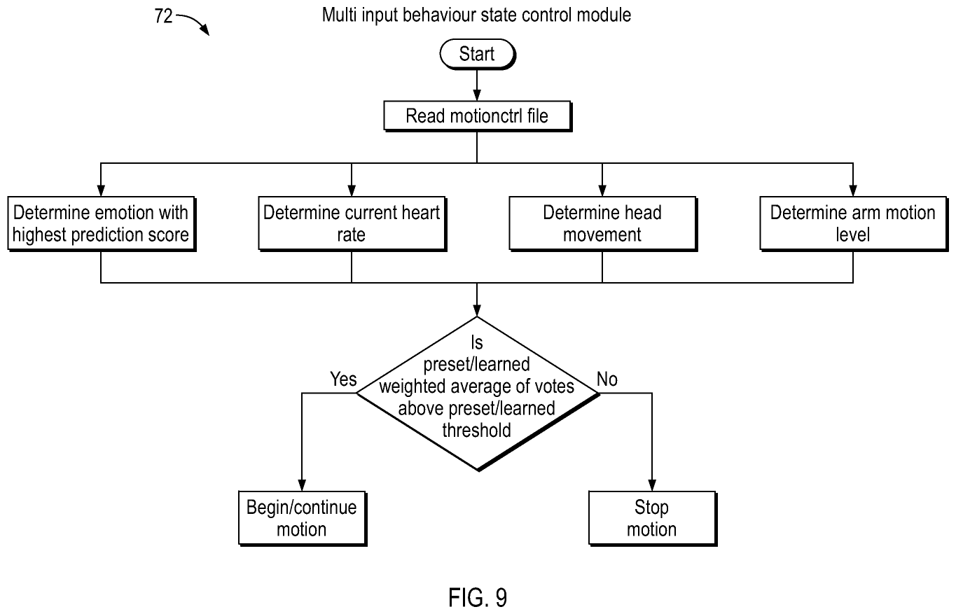

FIG. 9 outlines a detailed flow of the multi-input control module 72 using a weighted voting system to determine a final prediction of the patient's agitation state based on the aforementioned inputs. The weighted voting equation may be preset or learned to maximize the predictive accuracy of the device based on individual patient needs.

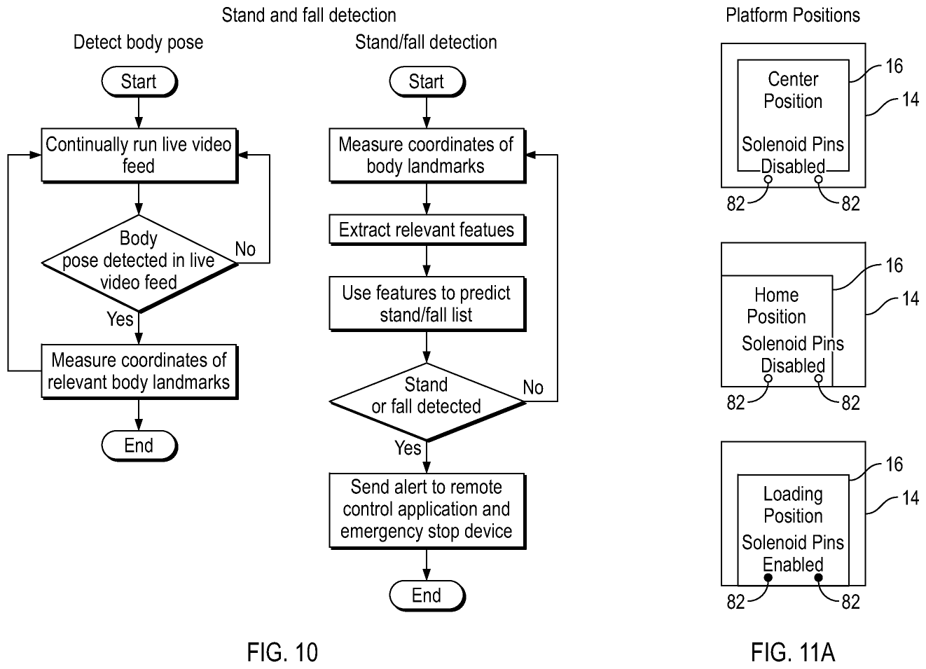

FIG. 10 outlines an algorithmic flow of a stand/fall detection module based on the continuous live video feed from the camera 20 and measurement of body posture. FIG. 10 processes the freeze-frame of relevant body landmarks to determine the patient's postural state. Detection of an excessive change in posture may trigger an emergency stop command to the state controller.

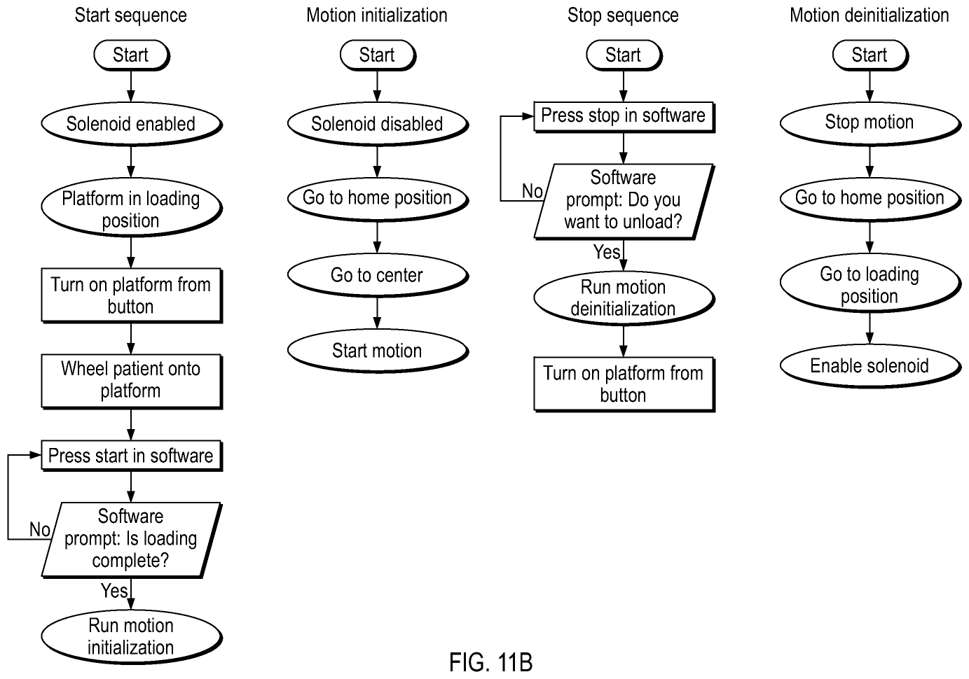

In some embodiments, the system 10 will include a platform locking mechanism comprising one or more locking pins or posts 82 on the base 14 which selectively engage with corresponding reinforced locking holes 84 on the bottom of the platform 16 (See FIGS. 2B, 2C, 3A and 11A). The locking posts 82 may comprise solenoid actuated posts or other similar type devices for selective actuation through the control system. The solenoids 82 may extend through openings 86 in the frame (FIG. 2B). When disabled, the posts are retracted back within the solenoid housing beneath the base frame covers. The locking mechanism will allow the operator to selectively lock the platform 16 in a loading position for safely loading and unloading a patient to and from the platform 16. As can be seen in FIG. 11A, the loading position of the platform 16 is generally forward and central to the base 14. FIG. 11B illustrates flow scenarios for loading and starting motion and stopping motion and unloading. In this regard, the control software will cycle the operator through a series of prompts for loading where the platform will move to the front and engage the posts 82 for loading, and once loaded, will move the platform to a home and then center position before initializing any motion sequence (See FIGS. 11A and 11B).

The embodiments disclosed herein have been discussed for the purpose of familiarizing the reader with the novel aspects of the invention. Although exemplary embodiments of the invention have been shown, many changes, modifications and substitutions may be made by one of ordinary skill in the art without necessarily departing from the spirit and scope of the invention as described in the following claims.

What is claimed is:

1. A therapeutic system for managing behavioral symptoms of neurological disorders comprising:
a motion device comprising a base and platform movable relative to the base, the platform having an upper surface configured to receive and support a seat thereon,
the motion device further comprising a motion actuation apparatus connected between the base and the platform effective for single-axis or biaxial oscillating motion of the platform relative to the base;
a camera device configured to acquire a continuous sequence of images of a patient seated on the platform; and
a feedback and control system comprising,
a data store encoded with content including a plurality of predetermined motion control profiles associated with managing predetermined agitated emotional states of a patient,
an analytics engine connected with the camera, the data store and the motion actuation apparatus,
said analytics engine receiving the continuous sequence of images from said camera, analyzing facial expression, head movements and/or physical movements within said images to determine in real-time an emotional state of the patient, and automatically altering operation of the motion actuation apparatus based on the emotional state of the patient.

2. The therapeutic system of claim 1, wherein the data store is further encoded with content including previously captured images of the patient in normal emotional states and agitated emotional states.

3. The therapeutic system of claim 1, wherein said analytics receives the continuous sequence of images form said camera, analyzes physical movements within said images to determine in real-time a standing state and/or a fallen state of the patient, and automatically altering or stopping operation of the motion actuation apparatus based on the state of the patient.

4. The therapeutic system of claim 1, further comprising a patient heart rate detection device connected with the feedback and motion control system,
said data store being further encoded with content including acceptable and out of range heart rate profiles, and
wherein an output of the heart rate detection device is used as a further input to the analytics engine for analyzing and determining in real-time an emotional state of the patient.

5. The therapeutic system of claim 4, wherein the heart rate detection device is a wrist worn device.

6. The therapeutic system of claim 1, further comprising a patient motion accelerometer device connected with the feedback and motion control system,
said data store being further encoded with content including acceptable and out of range motion profiles, and
wherein an output of the patient motion accelerometer device is used as a further input to the analytics engine for analyzing and determining in real-time an emotional state of the patient.

7. The therapeutic motion system of claim 6, wherein the patient motion accelerometer device is a wrist worn device.

8. The therapeutic system of claim 4, further comprising a patient motion accelerometer device connected with the feedback and motion control system,
said data store being further encoded with content including acceptable and out of range motion profiles, and
wherein an output of the patient motion accelerometer device is used as a further input to the analytics engine for analyzing and determining in real-time an emotional state of the patient.

9. The therapeutic system of claim 8, wherein the patient motion accelerometer device and the heart rate detection device are combined in a wrist worn device.

10. The therapeutic system of claim 1 further comprising an audio device connected with said feedback and control system,
said data store further encoded with auditory content,
said analytics automatically altering operation of the audio device to output said auditory content based on the emotional state of the patient.

11. The therapeutic system of claim 10, wherein the auditory content comprises music.

12. The therapeutic system of claim 3 further comprising an audio device connected with said feedback and control system,
said data store further encoded with auditory content,
said analytics automatically altering operation of the audio device to output said auditory content based on the emotional state of the patient.

13. The therapeutic system of claim 12, wherein the auditory content comprises music.

14. The therapeutic system of claim 4 further comprising an audio device connected with said feedback and control system,
said data store further encoded with auditory content,
said analytics automatically altering operation of the audio device to output said auditory content based on the emotional state of the patient.

15. The therapeutic system of claim 14, wherein the auditory content comprises music.

16. The therapeutic system of claim 6 further comprising an audio device connected with said feedback and control system,
said data store further encoded with auditory content,
said analytics automatically altering operation of the audio device to output said auditory content based on the emotional state of the patient.

17. The therapeutic system of claim 16, wherein the auditory content comprises music.

18. The therapeutic system of claim 1, further comprising a microphone connected with the feedback and motion control system,
wherein an output of the microphone is used as a further input to the analytics engine for analyzing and determining in real-time an emotional state of the patient.

19. The therapeutic system of claim 18, wherein the data store is further encoded with content including previously captured recordings of the patient in normal emotional states and agitated emotional states.

20. The therapeutic system of claim 1 further comprising a platform locking mechanism.
